(19) United States
(12) Patent Application Publication
(10) Pub. No.: US 2013/0218287 A1
(43) Pub. Date: Aug. 22, 2013

(54) Joint Socket and Method for Producing a Joint Socket

(71) Applicants: Andreas Meyenhofer, Schlattingen (CH); Martin Schmidt, Wangi (CH); Christian Gugler, Frauenfeld (CH); Daniel Nadler, Hettlingen (CH)

(72) Inventors: Andreas Meyenhofer, Schlattingen (CH); Martin Schmidt, Wangi (CH); Christian Gugler, Frauenfeld (CH); Daniel Nadler, Hettlingen (CH)

(73) Assignee: Jossi Holding AG, Islikon (CH)

(21) Appl. No.: 13/878,341

(22) PCT Filed: Aug. 31, 2011
(86) PCT No.: PCT/EP11/65016

§ 371 Date: Apr. 8, 2013

(30) Foreign Application Priority Data
Oct. 7, 2010 (EP) 01640/10

(51) Int. Cl. A61F 2/34

(52) U.S. Cl. CPC A61F 2/34
USPC 623/22.28

(57) ABSTRACT

The present invention relates to a joint socket (1) for a hip joint prosthesis, with a metal outer shell (2) and with a preferably ceramic inner shell (3). The outer shell (2) and the inner shell (3) are held together with a force fit by shrinkage and are connected to each other by at least one additional form-fit means (4; 5). The at least one form-fit means (4;5) is arranged on the periphery in the equatorial edge region of the joint socket in such a way that, as a result of the shrinkage stress, it forms a sealing operative connection between outer shell (2) and inner shell (3). The present invention further relates to a method for producing a joint socket (1), in which method a metal outer shell (2) is shrink-fitted onto a preferably ceramic inner shell (3), wherein at least one peripheral form-fit means (4;5) in the equatorial edge region of the outer shell (2) engages in a corresponding means of the inner shell (3).

The present invention relates to a joint socket according to the preamble of claim 1 and also to a method for producing a joint socket as claimed in claim 12.

Joint sockets in most cases consist of an inner shell, which is suitable for receiving a corresponding joint head, and an outer shell, which serves for anchoring into the bone. The inner shell here consists of tribologically suitable materials, such as certain metal alloys, polyethylene or ceramics, whereas the outer layer usually consists of an osteointegrable metal or a metal alloy. The two shells are in this case either connected to one another during production (non-modular sockets) or they are assembled during the operation (modular sockets).

In the case of non-modular sockets, the inner shell made of a plastic, such as polyethylene, polyoxyethylene (POM), polyether ether ketone (PEEK) or polyurethane (PU), can be inserted or adhesively bonded directly into a metallic outer shell, for example. Alternatively, the shells can also be connected to one another by means of a form fit.

EP 2 008 619 describes a joint socket in which the inner shell and the outer shell are connected by means of a press fit. The shrink fitting of the outer metal shell onto the ceramic inner shell is mentioned as a possible method.

U.S. Pat. No. 6,488,713 describes a method for connecting an inner shell made of polyethylene to a metallic outer shell in the case of a modular joint socket. In this case, the inner shell is joined by thermal expansion to the outer shell by using liquid nitrogen.

These methods and joint sockets have the disadvantage that the contact surface between the outer shell and the inner shell is sealed off to the outside to only a small extent. Body fluids can thereby pass between the shells and as a result abraded particles, which arise for example on insertion or during use of the joint, can escape therefrom. Furthermore, small volumes which are not hermetically sealed can lead to nucleation or to crevice corrosion with little exchange with the surroundings (drop in the local pH value).

It is accordingly an object of the present invention to avoid the disadvantages of the known joint sockets and in particular to provide a joint socket in which the two shells have a reliable fit in relation to one another and in which the contact surface between the shells is sealed off from the outside and inside. This object is achieved by a joint socket as claimed in claim 1.

The joint socket according to the invention for a hip joint prosthesis consists at least of a metallic outer shell and a preferably ceramic inner shell. The outer shell and the inner shell are held together in a force-fitting manner by means of a shrink-fit and are connected to one another by at least one additional form-fitting means. The at least one form-fitting means is arranged here peripherally in the equatorial edge region of the joint socket in such a manner that, by virtue of the shrinkage stress, it forms a sealing operative connection between the outer shell and the inner shell.

The combination according to the invention of a shrink-fit and form-fitting means achieves a reliable fit of the outer shell on the inner shell. In addition, the force fit prevents the two shells from moving in relation to one another.

The shrinkage stress which occurs presses the form-fit means into one another. This leads not only to a better fit of the form-fit means in relation to one another, but at the same time also leads to an additional sealing action arising between the form-fit means. Since the form-fit means are arranged in the equatorial edge region of the joint socket, the interface between the two shells is sealed off to the outside. On the one hand, this prevents the penetration of body fluids, for example blood, between the shells. On the other hand, the escape of abraded particles, which can arise between the shells upon operative insertion of the socket or by use of the implant, is prevented.

The shrink-fitting is carried out by heating the metallic outer shell and subsequently applying it to the preferably ceramic inner shell. On cooling, the outer shell shrinks and in the process combines with the inner shell in a force-fitting manner. Germs which may be present are additionally killed by the heating, with a germ-free space being formed between the shells.

The form-fitting means preferably comprises at least one peripheral rib or lip, which preferably engages into a peripheral groove.

The at least one peripheral rib can in this case be fitted on the inner side of the outer shell. Alternatively, the at least one rib can also be fitted peripherally on the outer side of the inner shell, however. The at least one rib can in this case have any suitable profile, for example dome-shaped or cuboidal. It is particularly preferable for the rib to have a triangular or wedge-shaped profile.

The peripheral rib preferably engages into a peripheral groove. The groove is in this case arranged on the respective other shell in such a manner that the rib can engage thereinto upon shrinkage in a fitting manner or with a slight oversize. The shrinkage stress presses the rib into the groove, with a sealing action being formed. Alternatively, the two shells can also have such a form that the form-fit elements initially snap into one another when the heated outer shell is placed on. Then, the shrinkage stress has the effect that the form-fit elements are pressed into one another in such a manner that a sealing action is formed by the additional plastic deformation of the form-fit elements.

As form-fitting means, the joint socket can furthermore also comprise a plurality of peripheral ribs, which preferably engage into peripheral grooves. In this case, all ribs can be arranged on the same shell. Alternatively, one or more ribs and one or more grooves can also be arranged on one shell.

The outer shell can alternatively also have a peripheral collar, which reaches over the bottom edge of the inner shell. The collar can in this case have such a form that it does not hinder the placement of the heated outer shell onto the inner shell. On subsequent cooling and shrinkage of the outer shell, the collar engages into the bottom edge of the inner shell.

In a preferred embodiment, an additional sealing element is arranged between a rib and the complementary groove. The shrinkage stress presses the rib into the sealing element, which increases the sealing action of the form fit. The additional sealing element is preferably configured as a sealing ring. The additional sealing element can consist of any suitable material, for example of a plastic such as polyethylene or else also of a metallic material.

The joint socket can alternatively have at least one additional, peripheral sealing element between the outer shell and the inner shell. The additional sealing element is in this case arranged outside the form-fitting means. The additional sealing element is preferably configured as a sealing ring. Alternatively, the additional sealing element can also be configured as a sealing lip, which is pressed against one of the shells by the shrinkage stress. Depending on the configuration, the sealing lip can buckle here or penetrate slightly into the shell. This results in an additional sealing effect.

The metallic outer shell consists of or comprises titanium, a titanium alloy, a steel alloy or a cobalt-chromium alloy. The outer shell can in this case have anchoring elements on its outer side, in order to promote the fastening in the bone. A multiplicity of different embodiments of such anchoring elements are known to a person skilled in the art. Furthermore, the outer shell can also have a coating.

The inner shell preferably consists of or comprises a ceramic material. Preferred ceramics here are aluminum oxide ceramics, zirconium oxide ceramics, aluminum-zirconium mixed ceramics, for example aluminum-toughened zirconia (ATZ) or zirconia-toughened aluminia (ZTA), and also carbide ceramics and nitride ceramics. Alternatively, the inner shell can also consist of a cobalt-based alloy or of an iron-based alloy. In this respect, however, the material should always have suitable tribological properties and also a high abrasion resistance, in order to serve as a sliding surface for the joint ball of a prosthesis.

Alternatively, the at least one form-fitting means can comprise a cutting edge on one shell, which digs into the other shell as a consequence of the shrinkage stress. In this embodiment, only one of the two shells has to be equipped with a form-fit element, since the complementary element is formed by the cutting edge digging in. This leads to a simpler production of the joint socket according to the invention and also to a higher sealing and locking action between the shells.

It is preferable here that the cutting edge is arranged on that shell of which the material has a higher degree of hardness than the material of the other shell. As a result, the cutting edge can dig into the other shell as a consequence of the shrinkage stress. It is preferable here that the outer shell consists of a metallic material and the inner shell consists of a ceramic or a polymer material. In this case, the ceramic material can have a higher degree of hardness than the metallic or polymer material. The corresponding material properties and advantageous material pairings are known to a person skilled in the art and are not described in more detail here.

Alternatively, a rib or lip can also be fitted on the shell consisting of the softer material and, upon shrinkage, is pressed onto the shell made of the harder material by the shrinkage stress. This rib or lip is thereby deformed, giving rise to a sealing action. In this embodiment, the rib or lip can also be pressed into a groove made peripherally in the shell made of harder material.

In this case, the harder material has a hardness of preferably 1200 to 2200 HV, whereas the softer material has a hardness of 300-400 HV.

The joint socket according to the invention is preferably used as part of a hip prosthesis. Alternatively, the joint socket according to the invention can also be used in other prostheses, for example for the shoulder or for ankle joints.

It is a further object of the present invention to present a suitable method which makes it possible to produce a joint socket having a good fit of the outer shell on the inner shell and in which the contact surface between the shells is sealed off from the outside and inside. This object is achieved by a method as claimed in claim 9.

In the method according to the invention, a metallic outer shell is shrink-fitted onto a preferably ceramic inner shell in such a manner that a force-fitting connection is formed, wherein at least one peripheral form-fitting means engages sealingly into a corresponding means of the inner shell in the equatorial edge region of the outer shell.

The additional engagement of at least one peripheral form-fit means additionally forms an operative connection between the two shells. The shrinkage stress which occurs presses the form-fit means onto a corresponding means on the other shell. The shrinkage stress has the effect that a sealing operative connection is formed.

In the method according to the invention, an additional sealing means can be introduced or applied by the shrinkage stress between the form-fitting means of the outer shell and the means of the inner shell and is pressed sealingly, by virtue of the shrinkage stress, between the form-fitting means of the outer shell and the means of the inner shell. It is preferable for the additional sealing means here to be a sealing ring, for example made of plastic or a metallic material, and also a sealing compound of plastic, metal or ceramic. The additional sealing means can additionally improve the sealing of the intermediate space between the inner shell and the outer shell.

In the method according to the invention, the outer shell is preferably firstly heated, in which case the shell expands. Then, the heated outer shell is placed onto a preferably ceramic inner shell. On subsequent cooling, the outer shell shrinks onto the inner shell, with the two shells being connected to one another in a force-fitting manner. The outer shell is heated here preferably to a temperature of 100° C. to 800° C.

Alternatively, in the method according to the invention, the inner shell can also be cooled, preferably to a temperature of −100° C. to −196° C. The cooling can be achieved, for example, by using liquid nitrogen. The inner shell contracts as a result of the cold. Then, the latter can be inserted into the outer shell. On subsequent heating, the inner shell expands into the outer shell, as a result of which the two shells are connected to one another in a force-fitting manner.

Further advantages and individual features of the invention become apparent from the following description of exemplary embodiments and from the drawings, in which:

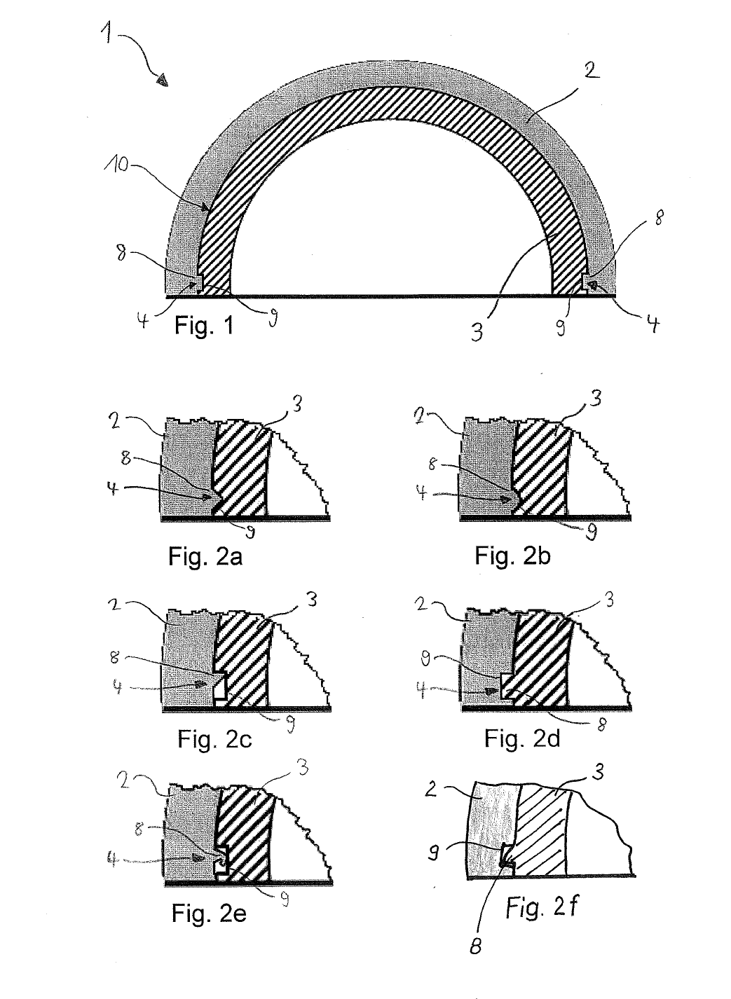

FIG. 1: shows a cross section through a joint socket according to the invention;

FIGS. 2a-2f: show enlarged illustrations of various alternative embodiments of the form-fit means in cross section;

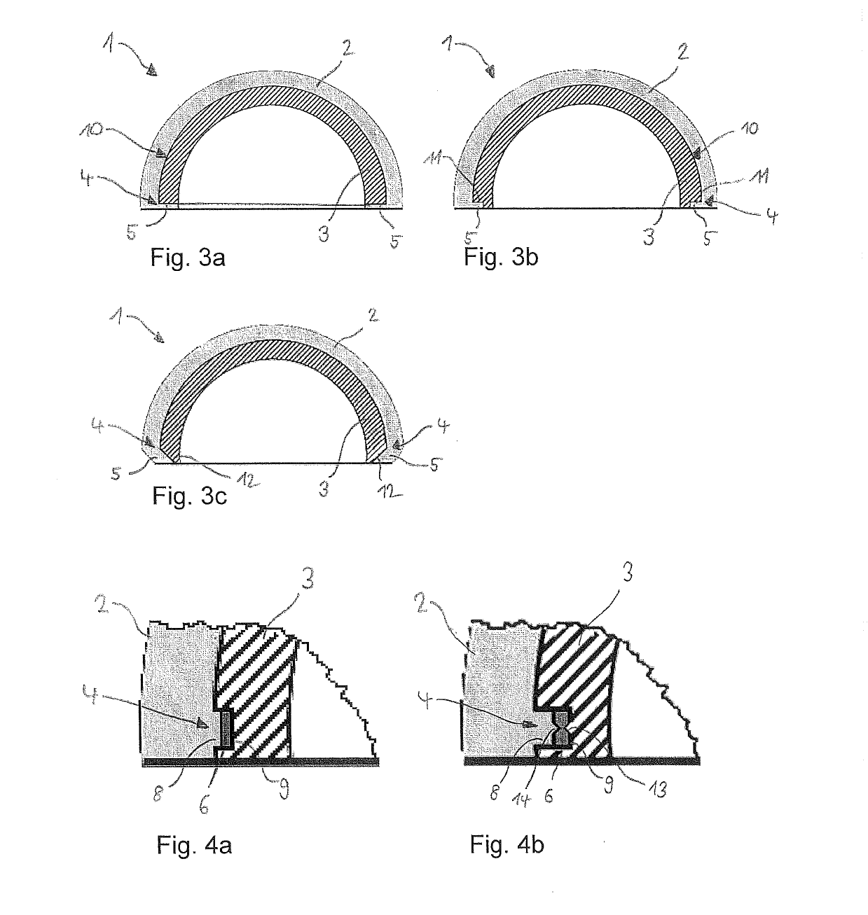

FIGS. 3a-3c: show sections through alternative embodiments of a joint socket, in which the outer shell has a collar;

FIGS. 4a-b: show enlarged sections of form-fit means with an additional sealing means; and

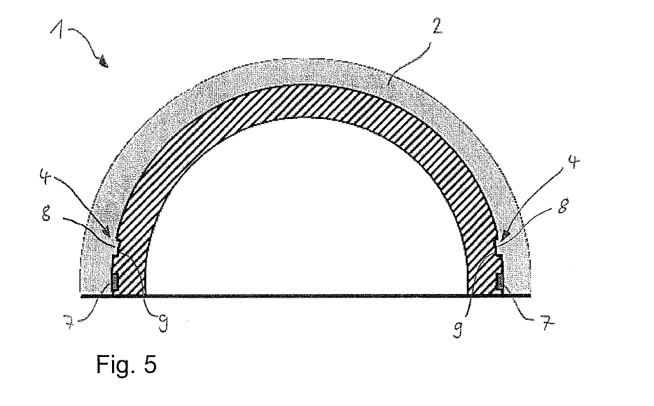

FIG. 5: shows a section through a further embodiment of a joint socket with an additional sealing means.

FIG. 1 shows a joint socket 1 according to the invention in section. The joint socket 1 consists of a metallic outer shell 2, which is shrink-fitted onto a preferably ceramic inner shell 3. The two shells are held together in a force-fitting manner by the shrink fitting. Additional peripheral form-fit means 4 make it possible to achieve an operative connection between the outer shell 2 and the inner shell 3. The form-fit means 4 are fitted in the equatorial edge region of the joint socket. In this embodiment, the form-fit means consist of a peripheral rib 8 on the inner side of the outer shell 2 and also of a peripheral groove 9 on the outer side of the inner shell 3. By virtue of the shrinkage stress, the operative connection additionally has a sealing action. The contact surface 10 between the inner shell 3 and the outer shell 2 is thereby sealed off to the outside and inside. This prevents the penetration of body fluids onto the contact surface 10. In addition, particles which form by abrasion at the contact surface 10 upon insertion of the joint socket or upon use of the joint are prevented from being able to escape into the body.

FIG. 2 shows alternative embodiments of the form-fit means 4, which consist of in each case a peripheral rib 8 and a peripheral groove 9. In the embodiment shown in FIG. 2a, the rib 8 on the inner side of the outer shell 2 has a triangular profile. The groove 9 on the outer side of the inner shell 3 likewise has a triangular profile and is dimensioned and arranged in such a manner that the rib 8 can engage thereinto with a precise fit or with an oversize. FIG. 2b shows an embodiment in which the peripheral rib 8 and the groove 9 have a round profile. In the embodiment shown in FIG. 2c, the peripheral rib 8 and the groove 9 have different profiles. The rib 8 here has a wedge-shaped profile, whereas the groove 9 has a quadrangular profile. The wedge-shaped rib 8 is in this case dimensioned and arranged in such a manner that a side face bears flush against a side face of the groove 9 on shrinkage. FIG. 2d shows a further embodiment of the form-fit means 4, in which the rib 8 lies peripherally on the inner shell 3. The rib 8 in this case engages into a groove 9 made peripherally on the inner side of the outer shell 2. FIG. 2e shows a further alternative embodiment of the form-fitting means 4, in which the rib 8 is pressed on account of the shrinkage stress against the outer face of the groove 9, with the rib 8 deforming. In this embodiment, the shell on which the rib 8 is fitted preferably consists of a material with a lower degree of hardness than the material of the shell on which the groove 9 is arranged.

FIG. 2f shows a variant in which the outer shell 2 likewise consists of softer material than the inner shell 3. The rib 8 firstly latches into the groove 9 in the outer shell and then presses into the groove bottom.

FIG. 3a shows a further embodiment of a joint socket 1 according to the invention. In this case, the outer shell 2 has a peripheral collar 5, which reaches over the bottom edge of the inner shell 3. This makes it possible to achieve a particularly good fit of the outer shell 2 on the inner shell 3. The sealing action arises here by virtue of the collar 5 being pressed against the bottom side of the inner shell 3. FIG. 3b shows an alternative embodiment, in which the collar 5 engages into a recess 11 arranged on the bottom edge of the inner shell. This embodiment of the form-fitting means reduces the abrasion at the contact surface 10, since micro-movements of the two shells 2, 3 against one another are prevented. In addition, the action of force on the outer shell 2 when the joint socket 1 is being implanted is avoided. FIG. 3c shows a further alternative embodiment, in which the peripheral collar 5 of the outer shell 2 acts on an obliquely running edge 12 of the inner shell 3.

FIG. 4 shows alternative embodiments of the form-fit means 4 in a sectional drawing and in an enlarged illustration. In the embodiment shown in FIG. 4a, an additional sealing element 6 is arranged between the rib 8 and the groove 9. The shrinkage stress presses the rib 8 into the additional sealing element 6, as a result of which a particularly good sealing action is achieved. The additional sealing means 6 is in this case preferably in the form of a sealing ring. FIG. 4b shows an alternative embodiment, in which the outer shell 2 and the inner shell 3 respectively have a lip 13, 14, which are pressed into the additional sealing element 6. An arrangement of this type additionally increases the sealing effect of the form-fitting means 4.

FIG. 5 shows a further embodiment of a joint socket 1 according to the invention. In this embodiment, an additional sealing means 7 is arranged between the outer shell 2 and the inner shell 3. The additional sealing means 7 is located outside the form-fit means 4. By way of example, the additional sealing means 7 can be in the form of a sealing ring. The outer shell 2 and the inner shell 3 preferably respectively have a peripheral groove suitable for receiving the additional sealing means 7.

1-15. (canceled)

16. A joint socket for a hip joint prosthesis, wherein a metallic outer shell and an inner shell are held together in a force-fitting manner by means of a shrink fit and are connected to one another by at least one additional form-fitting means, wherein the at least one form-fitting means is arranged peripherally in an equatorial edge region of the joint socket in such a manner that, by virtue of shrinkage stress, the form-fitting means forms a sealing operative connection between the outer shell and the inner shell.

17. The joint socket as claimed in claim 16, wherein the form-fitting means comprises at least one peripheral rib.

18. The joint socket as claimed in claim 16, wherein the form-fitting means comprises a plurality of peripheral ribs.

19. The joint socket as claimed in claim 18, wherein the peripheral rib or the plurality of peripheral ribs engage into a peripheral groove or into peripheral grooves, respectively.

20. The joint socket as claimed in claim 17, wherein the peripheral rib or the plurality of peripheral ribs engage into a peripheral groove or into peripheral grooves, respectively.

21. The joint socket as claimed claim 16, wherein the outer shell has a peripheral collar, which reaches over the bottom edge of the inner shell.

22. The joint socket as claimed in claim 16, wherein an additional sealing element is arranged between a groove and a rib.

23. The joint socket as claimed in claim 16, wherein the joint socket has at least one additional, peripheral sealing element between the outer shell and the inner shell.

24. The joint socket as claimed in claim 23, wherein the additional sealing element is a sealing ring.

25. The joint socket as claimed in claim 22, wherein the additional sealing element is a sealing ring.

26. The joint socket as claimed in claim 16, wherein the outer shell consists of a metal selected from the group consisting of titanium, titanium alloy, cobalt-chromium alloy and iron-based alloy.

27. The joint socket as claimed in claim 16, wherein the inner shell consists of a ceramic material.

28. The joint socket as claimed in claim 26, wherein the ceramic material is selected from the group consisting of aluminum oxide ceramic, zirconium oxide ceramic, aluminum-zirconium mixed ceramics, aluminum-toughened zirconia, zirconium-toughened alumina and also carbide and nitride ceramics.

29. The joint socket as claimed in claim 16, wherein the at least one form-fitting means comprises a cutting edge on one shell, which digs into the other shell as a consequence of the shrinkage stress.

30. The joint socket as claimed in claim 29, wherein one of said shells is harder than the other shell, and the cutting edge is arranged on the harder shell.

31. The joint socket as claimed in claim 16, wherein one of said shells is harder than the other shell, and a rib is arranged on the shell of lower hardness, said rib being deformed by the shrinkage stress.

32. A method for producing a joint socket, wherein a metallic outer shell is shrink-fitted onto an inner shell in such a manner that a force-fitting connection is formed, wherein at least one peripheral form-fitting means in the equatorial edge region of the outer shell engages sealingly into a corresponding means of the inner shell.

33. The method as claimed in claim 32, wherein an additional sealing means is introduced between the outer shell and the inner shell and is pressed sealingly, by virtue of shrinkage stress, between the form-fitting means of the outer shell and the means of the inner shell.

34. The method as claimed in claim 33, wherein the outer shell is heated and is then placed in the heated state onto the inner shell.

35. The method as claimed in claim 34, wherein the outer shell is heated to a temperature of 100° C. to 800° C.

36. The method as claimed in claims 32, wherein the inner shell is cooled and is then inserted in the cooled state into the outer shell.

37. The method as claimed in claims 36, wherein the inner shell is cooled to a temperature of −100° C. to −176° C.
